SEQUENCE LISTING (22 sequences)

SEQ ID NO: 1 - DNA, 2705 bp, Homo sapiens
  CDS (85)..(477)
ccgccgtagcgcgtcttgggtctcccggctgccgctgctgccgccgccgcctcgggtcgtggagccaggagcgacgtcaccgccatggcaggcatcaaagctttgattagtttgtcctttggaggagcaatcggactgatgtttttgatgcttggatgtgcccttccaatatacaacaaatactggcccctctttgttctatttttttacatcctttcacctattccatactgcatagcaagaagattagtggatgatacagatgctatgagtaacgcttgtaaggaacttgccatctttcttacaacgggcattgtcgtgtcagcttttggactccctattgtatttgccagagcacatctgattgagtggggagcttgtgcacttgttctcacaggaaacacagtcatctttgcaactatactaggctttttcttggtctttggaagcaatgacgacttcagctggcagcagtggtgaaaagaaattactgaactattgtcaaatggacttcctgtcatttgttggccattcacgcacacaggagatggggcagttaatgctgaatggtatagcaagcctcttgggggtattttaggtgctcccttctcacttttattgtaagcatactattttcacagagacttgctgaaggattaaaaggattttctcttttgggaaaagcttgactgatttcacacttatctatagtatgctttttgtggtgtcctgctgaatttaaatatttatgtgtttttcctgttaggttgattttttttggaatcaatatgcaatgttaaacactttttttaatgtaatcatttgcattggttaggaattcagaattccgccggctctattactggtcaagtacatcttttctcttaaaattatttagcctccattattacaaaaaattataaaaataagttttcagtcagtcaggatgacatcactcccaatgttatgcagacatacagacggttggcatacgttatagactgtatactcagtgcaaatatagctgcatttatacctcagaggggccaagtgttaatgcccatgccctccgttaagggttgttggttttactggtagacagatgttttgtggattgaaaattattttatggaattgctacagaggagtgcttttcttctcaattgttagaagaatttatgttaaactttaaggtaagggtgtaaaaacatttttgagataaggtttttatttatgtttattattgttagagtgagttgcaatgtgggaagaaatgacattgaaattccagtttttgaatcctgtttctatttataagtgaaatttgtgatctcctatcaacctttcatgttttaccctgttaaaatggacatacatggaaccactactgatgagggacagttgtatgtttgcatcatatatgccagaaaaccttcctctgcttcctccttttgacttatttggtatgttgtatatattacataaaataacttttcaaatatagtttaataacacttagaagtgtttacttacctggaaaataattgctatgccgtacattcagagtgccccctcccctgcaaggccttgccatgattaacaagtaacttgttagtcttacagataattcatgcattaacagtttaagatttagaccatggtaatagtagttcttattctctaaggttatatcatatgtaatttaaaagtatttttaagacaagtttcctgtatacctctgaactgttttgattttgagttcatcatgatagatctgctgtttccttataaaaggcatttgttgtgtgagttaatgcaaagtagccaagtccagctatatagcagcttcagaaacatacctgaccaaaaaattcccagtaaccaggcatgatcaatttatagtggtcgtttacatctaataattatcaggacttttttcaggagtgggttataaaaacattcaagttggtctgacagtattttgttaaggatatttgtttgtatgtttattcagtatacttacataaaaattatttcgccatcagccaaaactcagtaatcatgacagctgtctgttgttttatgaagtttatttctcaagaaaatgggaataaatttgggatttgttcagcttttttactaaagatgcctaaagccacaggttttattgcctaacttaagccatgacttttagatatgagatgacgggaagcaggacgaaatatcggcgtgtggctggagccttcccactggaggctgaaagtggcttgtggtattataatgttcagatttcaagaggaaggtgcaggtacacatgagttagagagctggtgagacagttgggaactctttgtgcttgtgatctactggactttttttttgcaggaagtgcattctctggtccttccctattttctgttctggatgtcagtgcagtgcactgctactgttttatccacttggccacagactttttctaacagctgcgtattatttctatatactaattgcattggcagcattgtgtctttgaccttgtatactagcttgacatagtgctgtctctgatttctaggctagttacttgagatatgaattttccatagaatatgcactgatacaacattaccattcttctatggaaagaaaacttttgatgatgaaacaataaagattttaaatatctaaattgttttcacacatgt

SEQ ID NO: 2 - protein, 131 aa, Homo sapiens
MAGIKALISLSFGGAIGLMFLMLGCALPIYNKYWPLFVLFFYILSPIPYCIARRLVDDTDAMSNACKELAIFLTTGIVVSAFGLPIVFARAHLIEWGACALVLTGNTVIFATILGFFLVFGSNDDFSWQQW

SEQ ID NO: 3 - protein, 131 aa, Mus musculus
MAGIKALISLSFGGAIGLMFLMLGCALPIYNQYWPLFVLFFYILSPIPYCIARRLVDDTDAMSNACKELAIFLTTGIVVSAFGLPVVFARAHLIEWGACALVLTGNTVIFATILGFFLVFGSNDDFSWQQW

SEQ ID NO: 4 - DNA, 1114 bp, Homo sapiens
  CDS (71)..(463)
gtctggcttgggcaggctgcccgggccgtggcaggaagccggaagcagccgcggccccagttcgggagacatggcgggcgttaaagctctcgtggcattatccttcagtggggctattggactgacttttcttatgctgggatgtgccttagaggattatggcgtttactggcccttattcgtcctgattttccacgccatctcccccatcccccatttcattgccaaaagagtcacctatgactcagatgcaaccagtagtgcctgtcgggaactggcatatttcttcactactggaattgttgtttctgcctttggatttcctgttattcttgctcgtgtggctgtgatcaaatggggagcctgcggccttgtgttggcaggcaatgcagtcattttccttacaattcaagggtttttccttatatttggaagaggagatgattttagctgggagcagtggtagcactttattctgattacagtgcattgaatttcttagaactcatactatctgtatacatgtgcacatgcggcattttactatgaaatttaatatgctgggttttttaatacctttatatatcatgttcactttaagaaagacttcataagtaggagatgagttttattctcagcaaatagacctgtcaaatttagattatgttactcaaattatgttacttgtttggctgttcatgtagtcacggtgctctcagaaaatatattaacgcagtcttgtaggcagctgccaccttatgcagtgcatcgaaaccttttgcttggggatgtgcttggagaggcagataacgctgaagcaggcctctcatgacccaggaaggccggggtggatccctctttgtgttgtagtccatgctattaaaagtgtggcccacagaccaagagcctcaacatttcctagagccttattagaaatgcagaatctgaagccccactctggacccaggacattttgatgagatccaaaggagttgtatgcacatgaaagtttgagaagcatcatcatagagaagtaaacatcacacccaacttccttatctttccagtggctaaaccacttaacctctctgggtgttacctgctcatttgttta

SEQ ID NO: 5 - protein, 131 aa, Homo sapiens
MAGVKALVALSFSGAIGLTFLMLGCALEDYGVYWPLFVLIFHAISPIPHFIAKRVTYDSDATSSACRELAYFFTTGIVVSAFGFPVILARVAVIKWGACGLVLAGNAVIFLTIQGFFLIFGRGDDFSWEQW

SEQ ID NO: 6 - protein, 131 aa, Mus musculus
MAGVKALVALSFSGAIGLTFLMLGCALEDYGVYWPLFVLIFYVISPIPYFIAKRVTYDSDATSSACRELAYFFTTGIVVSAFGLPVVLARVDVIKWGACGLVLAGNAVIFLTIQGFFLVFGRGDDFSWEQW

SEQ ID NO: 7 - protein, 9 aa, Artificial Sequence
  Description of Artificial Sequence Synthetic peptide
YPYDVPDYA

SEQ ID NO: 8 - protein, 10 aa, Artificial Sequence
  Description of Artificial Sequence Synthetic peptide
EQKLISEEDL

SEQ ID NO: 9 - protein, 11 aa, Artificial Sequence
  Description of Artificial Sequence Synthetic peptide
YTDIEMNRLGK

SEQ ID NO: 10 - protein, 6 aa, Artificial Sequence
  Description of Artificial Sequence Synthetic 6xHis tag
HHHHHH

SEQ ID NO: 11 - protein, 8 aa, Artificial Sequence
  Description of Artificial Sequence Synthetic peptide
DYKDDDDK

SEQ ID NO: 12 - DNA, 26 bp, Artificial Sequence
  Description of Artificial Sequence Synthetic primer
caaatactggcccctctttgttcatt

SEQ ID NO: 13 - DNA, 27 bp, Artificial Sequence
  Description of Artificial Sequence Synthetic primer
tcagtaatttcttttcaccactgctgc

SEQ ID NO: 14 - DNA, 20 bp, Artificial Sequence
  Description of Artificial Sequence Synthetic primer
agcagccgcggccccagttc

SEQ ID NO: 15 - DNA, 21 bp, Artificial Sequence
  Description of Artificial Sequence Synthetic primer
aaggccgcaggctccccattt

SEQ ID NO: 16 - DNA, 21 bp, Artificial Sequence
  Description of Artificial Sequence Synthetic primer
cacactgtgcccatctacgag

SEQ ID NO: 17 - DNA, 21 bp, Artificial Sequence
  Description of Artificial Sequence Synthetic primer
cgtggtggtgaagctgtagcc

SEQ ID NO: 18 - DNA, 19 bp, Artificial Sequence
  Description of Artificial Sequence Synthetic primer
gtgaaggtcggagtcaacg

SEQ ID NO: 19 - DNA, 21 bp, Artificial Sequence
  Description of Artificial Sequence Synthetic primer
catgggtggaatcatattgga

SEQ ID NO: 20 - DNA, 26 bp, Artificial Sequence
  Description of Artificial Sequence Synthetic primer
acgaattcaccgccatggcaggcatc

SEQ ID NO: 21 - DNA, 27 bp, Artificial Sequence
  Description of Artificial Sequence Synthetic primer
actctagaccactgctgccagctgaag

SEQ ID NO: 22 - protein, 9 aa, Artificial Sequence
  Description of Artificial Sequence Synthetic peptide
PYDVPDYAY

(19) United States
(12) Patent Application Publication
(10) Pub. No.: US 2006/0068429 A1
(43) Pub. Date: Mar. 30, 2006

(54) LEPROTL1 and OB-RGRP genes for screening compounds that act on the gain or loss of weight or on diabetes in a human or animal subject

(75) Inventors: Bernard Bailleul, Lille (FR); Yves Rouille, Wasquehal (FR); Karin Seron, Saint-Omer (FR); Sandrine Belouzard, Lille (FR)

Correspondence Address: IP GROUP OF DLA PIPER RUDNICK GRAY CARY US LLP, 1650 MARKET ST, SUITE 4900, PHILADELPHIA, PA, 19103, US

(73) Assignee: Centre National de la Recherche Scientifique - CNRS, an organization of France, Paris Cedex (FR)

(21) Appl. No.: 11/221,275

(22) Filed: Sep. 7, 2005

Related U.S. Application Data
(63) Continuation of application No. PCT/FR04/00572, filed on Mar. 10, 2004.

(30) Foreign Application Priority Data
Mar. 10, 2003 (FR) 03/02931

(51) Int. Cl. C12Q 1/68; G01N 33/53

(52) U.S. Cl. 435/6; 435/7.1

(57) ABSTRACT

A method for identifying compounds acting on weight gain or weight loss or on diabetes in a human or animal subject including measuring effect of the compounds on (a) expression of at least one of genes LEPROTL1 and OB-RGRP or a part of them and/or (b) intracellular transport up to a cellular membrane and/or (c) presence at the cellular membrane level and/or (d) internalization from the membrane of proteins coded by at least one of these genes or part of them.

### RELATED APPLICATION

This is a continuation of International Application No. PCT/FR2004/000572, with an international filing date of Mar. 10, 2004 (WO 2004/080272, published Sep. 23, 2004), which is based on French Patent Application No. 03/02931, filed Mar. 10, 2003.

### FIELD OF THE INVENTION

This invention relates to diagnosing, prevention and treatment of obesity or weight loss associated or not associated with hormonal disorders and also diabetes in humans or in animals. The invention relates more particularly to a novel method of screening compounds useful for treating or preventing obesity or weight loss or diabetes in a human or animal subject based on the use of the LEPROTL1 and OB-RGRP genes and the traffic of proteins coded by these genes, respectively endospanin and OB-RGRP.

### BACKGROUND

A family of small hydrophobic proteins has been described whose human and murine representatives called OB-RGRP (leptin receptor gene-related protein) come from an alternative splicing at the level of the locus of the receptor of leptin (OB-R), but which have no similarity of sequence with the isoforms of OB-R and whose function has not been elucidated (B. Bailleul et al., 1997, Nucleic Acids Res., 25, 2752-2758). The existence of a protein called “endospanin” has also been reported that is very homologous to OB-RGRP, coded by the LEPROTL1 gene (leptin receptor overlapping transcript-like 1) (Y. Huang et al, 2001, Biochim. Biophys. Acta, 1517, 327-331). At the cellular level these proteins with four transmembrane areas are localized in membrane areas rich in cholesterol and glycol-sphingolipids (K. Seron et al.; poster congress/convention). These proteins are expressed in a large number of tissues and especially in the majority of the peripheral tissues.

### SUMMARY OF THE INVENTION

This invention relates to a method for identifying compounds acting on weight gain or weight loss or on diabetes in a human or animal subject including measuring effect of the compounds on (a) expression of at least one of genes LEPROTL1 and OB-RGRP or a part of them and/or (b) intracellular transport up to a cellular membrane and/or (c) presence at the cellular membrane level and/or (d) internalization from the membrane of proteins coded by at least one of these genes or part of them.

### BRIEF DESCRIPTION OF THE DRAWINGS

Other advantages and characteristics of the invention will appear from the following examples that refer to the attached drawings.

FIG. 1 shows the sequence of the DNAc of the LEPROTL1 gene (SEQ ID NO. 1) in which the reading frame is underlined.

FIG. 2 shows the alignment of the sequence of diazoxide and of human and murine OB-RGRP. The four transmembrane areas of these proteins are indicated in line 1. FIG. 2 discloses SEQ ID NOS. 5, 6, 2 and 3, respectively, in order of appearance.

FIG. 3 shows HeLa cells with double marking in indirect immunofluoresence expressing endospanin-HA-B1, incubated in the presence of antibodies of mouse anti-HA. Green fluorescence reveals the internalized anti-HA antibodies (A and C). Red fluorescence reveals endospanin, —HA-B1 and endogenous in the cells (B and C).

FIG. 4 shows the quantity of leptin receptor on the surface relative to the total quantity of cellular leptin normalized 100% for the control of HeLa cells co-infected with two recombinant adenoviruses in order to express a constant quantity of OBRa and increasing quantities of endospanin (multiplicity of infection from 0 to 80).

FIG. 5 shows the expression of RNAm of OB-RGRP (left) and of LEPROTL1 (right) in line MCF7 during treatment with oestradiol/estradiol and with progesterone by PCR in real time.

FIG. 6 shows the distribution and the intensity of expression of RNAm, OB-RGRP (left) and of LEPROTL1 (right) in the brain and fetus of the mouse.

FIG. 7 shows that the overexpression of the protein OB-RGRP induces a dose-dependent diminution of the expression on the surface of the leptin receptor.

### DETAILED DESCRIPTION

We have now discovered the role of OB-RGRP and endospanin on the expression of OB-R on the surface of cells. Thus, the study of the expression of genes coding these proteins shows a co-expression of these two genes, in particular in the cells of organs expressing OB-R (J. Mercer at al., 2000, J. Neuroendocrinol., 12, 649-655). We have now also demonstrated that the gene coding endospanin participates in the intracellular traffic of OB-R and, thus, regulates the rate of OB-R at the level of the external membrane of the self. We therefore believe that these genes play a part in the sensitivity of the cell to leptin and their regulation permits defining a novel pharmacological strategy for intervening in a sensitivity to leptin by augmenting or reducing expression of OB-R at the level of the cellular membrane and for treating or preventing weight loss or weight gain by individual humans or animals.

We can now explain sexual dimorphism at the level of the adipose/fatty mass rate and the rate of circulating leptin. We have now also demonstrated that LEPROTL1 and OB-RGRP are regulated by certain sexual hormones. More particularly, a hormonal regulation of these two genes permits taking into account sexual polymorphism and variations during the life of a woman, the rate of circulating leptin and therefore the distribution and the gaining of adipose mass.

This invention relates to a method for identifying compounds acting on weight gain or weight loss or on diabetes in a human or animal subject comprising measuring the effect of these compounds on (a) expression of at least one of the genes LEPROTL1 and OB-RGRP or part of them and/or (b) intracellular transport up to the cellular membrane and/or (c) presence at the cellular membrane level of proteins and/or (d) internalization from the membrane of proteins coded by at least one of these genes or part of them.

In fact, the study of the intracellular traffic of endospanin permitted the demonstration that this protein passes to the cellular surface and is then endocytosed, i.e., incorporated into the cell, then degraded into the lysomes. It is believed that OB-RGRP participates in the same intracellular traffic.

More particularly, the method comprises (i) contacting a compound to be tested with cells expressing at least one of the LEPROTL1 and OB-RGRP genes or fragments of them, then (ii) measuring the quantity of proteins coded by at least one of these genes or part of them present at the level of the membrane of these cells and/or their internalization.

The endospanin and/or OB-RGRP or part of them is/are advantageously marked in such a manner as to be able to be detected and thus realize the measuring of the method of the invention. Every type of marking can be envisioned within the framework of the method of the invention. However, a marking of the peptide tag type is preferred.

The method of the invention can be implemented in vitro or in vivo in an animal.

The method of the invention can also comprise measuring the expression and/or the transport advantageously up to the surface of the membrane of the cells of OB-R or, in the case of an animal, the measuring of the circulating leptin.

The cellular or animal models can be of the type either expressing LEPROTL1 and/or OB-RGRP or fragments of them in an endogenous manner, that is, genetically modified to express these LEPROTL1 and/or OB-RGRP or fragments of them in the form of recombinant proteins.

A human model such as HeLa, 293 or HepG2 or murine model such as 3T3 can be cited as examples of cellular models naturally expressing LEPROTL1 and/or OB-RGRP, and primary cultures could also be used. Any type of cell can be used because the majority of the lines and tissues express endospanin and OB-RGRP.

The mouse, rat and rabbit can be cited as representative examples of animal models naturally expressing LEPROTL1 and/or OB-RGRP.

However, an implementation of the invention on cells transformed to express all or part of at least one of the genes LEPROTL1 and/or OB-RGRP is preferred. This advantageously concerns cells in culture. However, the invention may also be implemented in animals obtained by transgenesis in accordance with techniques described in the literature from LEPROTL1 and/or OB-RGRP or fragments of them.

Cells in culture that were genetically modified for expressing at least one of LEPROTL1 and/or OB-RGRP or fragments of them are preferred.

The sequences of DNAc of human LEPROTL1 and of human and murine endospanin are given in the attached sequence list under the respective numbers SEQ ID NO. 1, SEQ NO. 2 and SEQ ID NO. 3.

The sequences of DNAc, human OB-RGRP and human and murine protein are given in the attached sequence list under the respective numbers SEQ ID NO. 4, SEQ ID NO. 5 and SEQ ID NO. 6.

These genetically modified cells are prepared by techniques well known in the art implementing expression vectors.

Of course, this can involve human or murine forms or forms of any other species if they advantageously have at least 80% homology with human LEPROTL1 and OB-RGRP.

This can also involve fragments or sequences modified by the addition, deletion or change of one or several nucleotides of these genes from when the protein coded by its modified fragments or sequences can be transported to the cellular membrane and then advantageously be internalized.

A preferred aspect comprises expressing a modified form of LEPROTL1, OB-RGRP or fragments of them in such a manner as to produce marked proteins. The marking advantageously comprises a peptide tag. The term “modified form of these genes” also denotes genes coding for truncated proteins, that is, deleting one or several amino acids, useful in particular for peptide marking.

As previously indicated, endospanin and/or OB-RGRP comprise(s) four transmembrane areas. Thus, by way of a preferred example, a fragment and a modified form of LEPROTL1 or OB-RGRP codes for a modified protein comprising at least two of the four transmembrane areas and a peptide marker in the form of fusion proteins.

The marker of endospanin and/or OB-RGRP or their fragments can be identical or different. The following can be cited as examples of generally used peptide markers:

| | | |
|---|---|---|
| HA of sequence | YPYDVPDYA, | (SEQ ID NO. 7) |
| c-myc: sequence | EQKLISEEDL, | (SEQ ID NO. 8) |
| VSV-G: sequence | YTDIEMNRLGK, | (SEQ ID NO. 9) |
| His₆: sequence | HHHHHH, | (SEQ ID NO. 10) |
| FLAG: sequence | DYKDDDDK. | (SEQ ID NO. 11) |

Insertion of a peptide marker into the complete protein can be carried out at any level of the protein or a fragment of it when it does not modify the expression of the protein at the cellular membrane and advantageously also its internalization.

Thus, in the case of endospanin and OB-RGRP the sites between the amino acids of positions 25 to 35 and of positions 82 to 100 of SEQ ID NO. 2 and SEQ ID NO. 5, that is, the two extracellular loops, can be cited. The extension in the N or C terminal by a peptide marker on a truncated protein is also possible by ensuring that the deletion positions the terminal end and the marker in luminal/extracellular position.

The reading frame should be integrated into an expression vector of the plasmid (pCI/pCIneo, Promega, pcDNA3, Invitrogen) type or virus (adeno, retro, vaccine) type for expressions that are transitory or stable.

As previously indicated, endospanin and/or OB-RGRP or part of them can be marked and a marking of the peptide tag type is preferred. The measuring can then be carried out with the aid of an antibody directed against the peptide marker, integrated, e.g., in one of the two luminal/extracellular loops of endospanin, OB-RGRP or fragments of them or in a prolongation of these loops in the truncated forms of the proteins by placing the antibody in contact with the cells.

The method can also be carried out on unmarked endospanin and/or OB-RGRP. Antibodies directed against one of the two luminal/extracellular loops of endospanin or of OB-RGRP permit detection of the endogenous protein by being placed in contact with the cells.

By way of example, the position of the polypeptides for obtaining these specific, preferably monoclonal antibodies for identifying endogenous endospanin and OB-RGRP at the membrane level is situated between the amino acids at positions 25 to 35 and the amino acids at positions 82 to 100 of SEQ ID NO. 2 and SEQ ID NO. 5.

The measuring can then comprise the realization of a secondary antibody in accordance with techniques well known in the art.

Several techniques can be cited for quantifying the proteins that can be used within the framework of the invention:
ELISA: Secondary coupled with peroxidase, with alkaline phosphatase or some other enzyme, quantifiable and automatable colored reaction,
The Western blot,
Radioactive counting (iodine),
Immunofluorescence: Fluorescent secondary antibody useful for measuring the morphology.

Other measuring techniques can be realized such as, e.g.:
Biotinylation of surface and streptavidin,
Iodination of surface and immuno-precipitation.

The effect of the compound to be tested in accordance with the method can be realized by a comparison of the measurings on the cells with and without a placing in contact with this compound. This effect can be measured after or during a determined time of placing the cells in contact with this compound.

The specificity of the compound to be tested vis-à-vis a) expression of at least one of the genes LEPROTL1 and OB-RGRP and/or (b) intracellular transport up to the cellular membrane and/or (c) presence at the level of the cellular membrane of the proteins and/or (d) internalization from the membrane of the proteins coded by at least one of these genes or a part of them can be determined by measuring the effect of the treatment on other genes or proteins.

Thus, the effect of the compound to be tested on the intracellular traffic of endospanin and/or OB-RGRP is also measured with the aid of an antibody directed against any surface membrane protein, a part or a marker of the latter.

The transferin receptor can be cited as a preferred example.

The invention also relates to a pharmaceutical composition for the prevention or treatment of weight gain or weight loss or of diabetes, comprising at least one compound capable of modifying the expression of at least one of the genes LEPROTL1 and OB-RGRP or part of them, and/or (b) intracellular transport up to the cellular membrane, and/or (c) presence at the level of the cellular membrane, and/or (d) internalization from the membrane of proteins coded by at least one of these genes or part of them. Such a compound is advantageously an antagonist or an agonist of at least one of the proteins endospanin and OB-RGRP.

The invention also relates to the diagnosis of weight gain or loss in a human or animal subject comprising measuring and advantageously comparing with at least one control the expression of at least one of the genes LEPROTL1 and OB-RGRP or part of them, and/or (b) intracellular transport up to the cellular membrane, and/or (c) presence at the level of the cellular membrane, and/or (d) internalization from the membrane of proteins coded by at least one of these genes or part of them.

### EXAMPLE 1

### Transport of a Marked Version of the Endospanin of the Membrane on the Cytoplasm

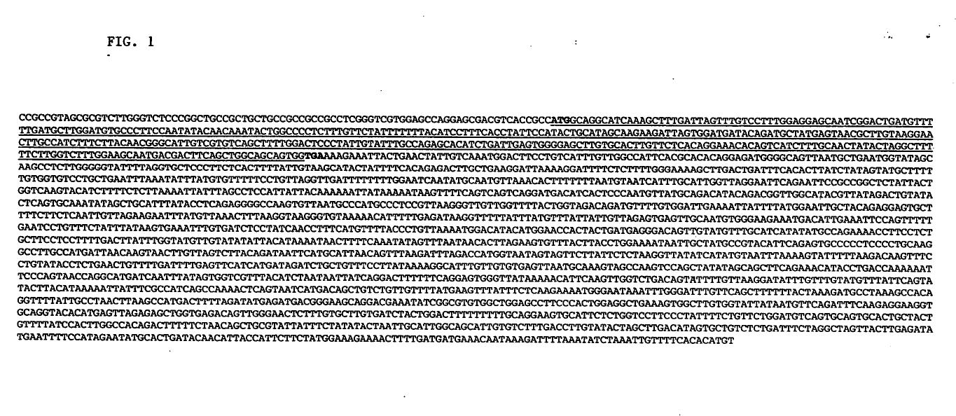

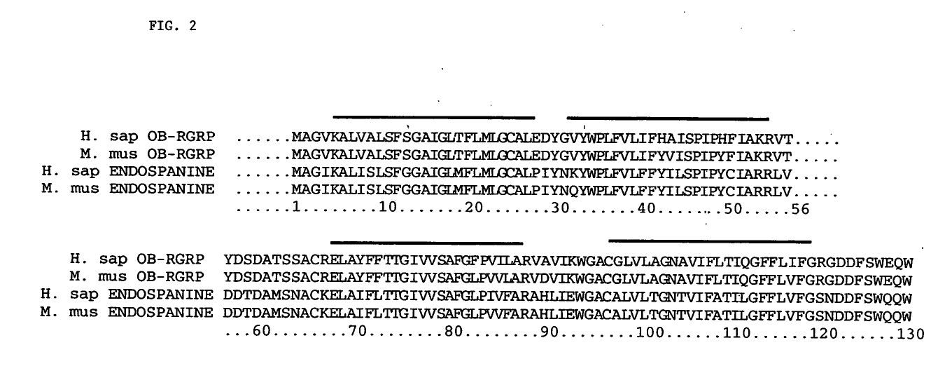

An epitope (or tag) HA (9 amino acids: PYDVPDYAY; SEQ ID NO. 22) was introduced by directed mutagenesis into the first extracellular loop (B1) of endospanin between residues Y30 and N3a (FIGS. 1 and 2). Linking residues were included on each side of the tag (respectively GAS and GA). HeLa cells previously cultivated on glass slides were transfected with a plasmid permitting the expression of the protein marked in this manner, endospanin. Twenty-four hours after transfection, the cells were incubated two hours in a culture environment containing an anti-HA monoclonal antibody. The cells were then rinsed, then incubated in a culture environment lacking antibodies. The cells were fixed in a solution of 3% paraformaldehyde. Internalized anti-HA antibody was revealed with the aid of an anti-IgG antibody of a mouse marked at alexa-488 after previous permeabilization of the cells by an incubation in a solution containing 0.1% triton X-100. Endospanin was revealed with the aid of an anti-endospanin rabbit antiserum against the dodecapeptide of the C-terminal part and by a rabbit anti-IgG antibody marked at alexa-594. The preparations were observed on an immuno-fluorescent microscope.

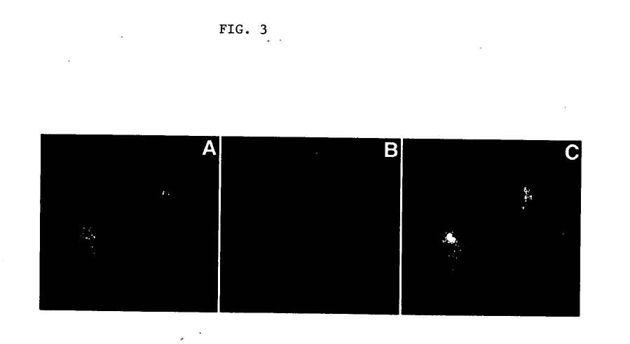

A punctuate cytoplasmic signal was observed, indicating that anti-HA antibodies were internalized from the plasma membrane toward endosomal intro-cytoplasmic compartments (FIGS. 3A and 3C). A total marking was performed on the same cells after fixation with the aid of an antibody directed against the C-terminal part of endospanin to show that this marking corresponded well to internalized proteins. The pattern of this total marking differs considerably from that corresponding to the internalized protein on the one hand and reproduces the pattern of endogenous endospanin (FIGS. 3B and 3C). This demonstrates that the marking with anti-HA antibodies results from an internalization of the surface protein and indicates that the addressing on the cellular surface of the protein marked with an HA epitope does not result from an overexpression artifact and that it therefore probably behaves like the endogenous protein.

The same experiment was carried out as a control in parallel with endospanin carrying the same HA epitope in its cytosolic loop (between residues M62 and S63). This loop was not exposed to the outside of the cell. It is consequently inaccessible to the anti-HA antibodies present in the culture environment. As foreseen, this construction does not permit internalization of the anti-HA antibody to be observed.

These results demonstrate that the endospanin into which an HA epitope was inserted (in its first extracellular loop) is addressed to the plasma membrane of the cell, then internalized in the endosomes. Four to five hours after internalization, the signal corresponding to the anti-HA antibody could no longer be detected. This loss of marking shows that the endospanin was transported to intracellular compartments, probably lysosomes, where the internalized antibodies were degraded.

### EXAMPLE 2

### Modulation of the Expression of the Leptin Receptor on the Surface of Cells by Endospanin

Recombinant adenoviruses expressing a short isoform (OB-Ra) of murine OB-R and human endospanin were constructed by homologous recombination. OB-R expressed by this viral vector comprises an HA epitope on its N-terminal end. HeLa cells were co-infected with the virus expressing the receptor and increasing doses of virus expressing endospanin. The surface proteins were biotinylated at 4° C. after 24 hours of expression. The cells were then lysed, then the cellular lysates clarified by centrifugation. The relative layers of biotinylated receptors (surface) were quantified by immunoblot from material purified by spheres of streptavidin agarose and revelation by immunoblot after separation of the proteins by electrophoresis in polyacrylamide gel. The total receptors (surface+internal) were quantified in parallel by the same method from a tenth of each cellular lysate. OB-Ra was revealed with an anti-HA anti-body. The same immunoblots were then revealed by an antibody directed against the receptor of transferrin. The quantities of surface receptors were expressed by the ratio of the quantity of receptor in the biotinylated fraction to the quantity of receptor present in the total cellular lysates.

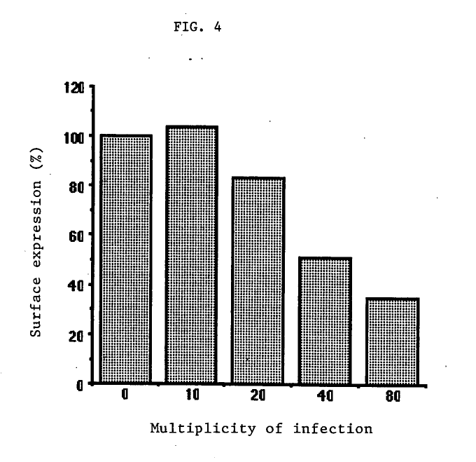

The surface expression of OB-R is affected by the overexpression of endospanin. The overexpression of endospanin induces a dose-dependent diminution of the surface expression of OB-RGRP (FIG. 4) with no impact on the surface expression of the receptor of transferrin. These results demonstrate the specificity of the effects observed on the surface expression of OB-RGRP and suggest that endospanin participates in the regulation of the surface expression of this receptor.

### EXAMPLE 3

### Hormonal Regulation of LEPROTL1 and OB-RGRP

Expression of the genes LEPROTL1, OB-RGRP, b-actin and GAPDH was studied by quantitative PCR in real time on a LightCycler apparatus (Roche) in the human line of breast cancer MCF7 subjected or not subjected to a treatment either by progesterone or by estradiol. The MCF7 cells cultivated in a DMEM environment without phenol red at 37° C. under 5% CO₂ are deprived of serum for one night, then treated (1 mM progesterone or estradiol) for 6 hours. The total RNA's were prepared using the following kits: QIAGEN, Rneasy®. The cDNA's (2 mg of RNA in a volume of 25 μL) were obtained with the aid of reverse transcriptase (M-MLV, Promega) and Random oligonucleotides p (dN6) (Roche) as primers and by the method recommended by the supplier.

We used the kit and the materials of the Roche company (LightCycler-FastStart DNA Master SYBR Green I, LightCycler Capillaries). The quantifications were carried out with DNAc's corresponding to 100 ng RNA by the method recommended by the supplier.

The oligonucleotides for the PCR's in real time are the following:

| | |
|---|---|
| LEPROTL1: | |
| 5′CAAATACTGGCCCCTCTTTGTTCATT3′ | (SEQ ID NO. 12) |
| 5′TCAGTAATTTCTTTTCACCACTGCTGC3′ | (SEQ ID NO. 13) |
| OB-RGRP: | |
| 5′AGCAGCCGCGGCCCCAGTTC3′ | (SEQ ID NO. 14) |
| 5′AAGGCCGCAGGCTCCCCATTT3′ | (SEQ ID NO. 15) |
| β-Actine: | |
| 5′CACACTGTGCCCATCTACGAG3′ | (SEQ ID NO. 16) |
| 5′CGTGGTGGTGAAGCTGTAGCC3′ | (SEQ ID NO. 17) |
| GAPDH: | |
| 5′GTGAAGGTCGGAGTCAACG3′ | (SEQ ID NO. 18) |
| 5′CATGGGTGGAATCATATTGGA3′ | (SEQ ID NO. 19) |

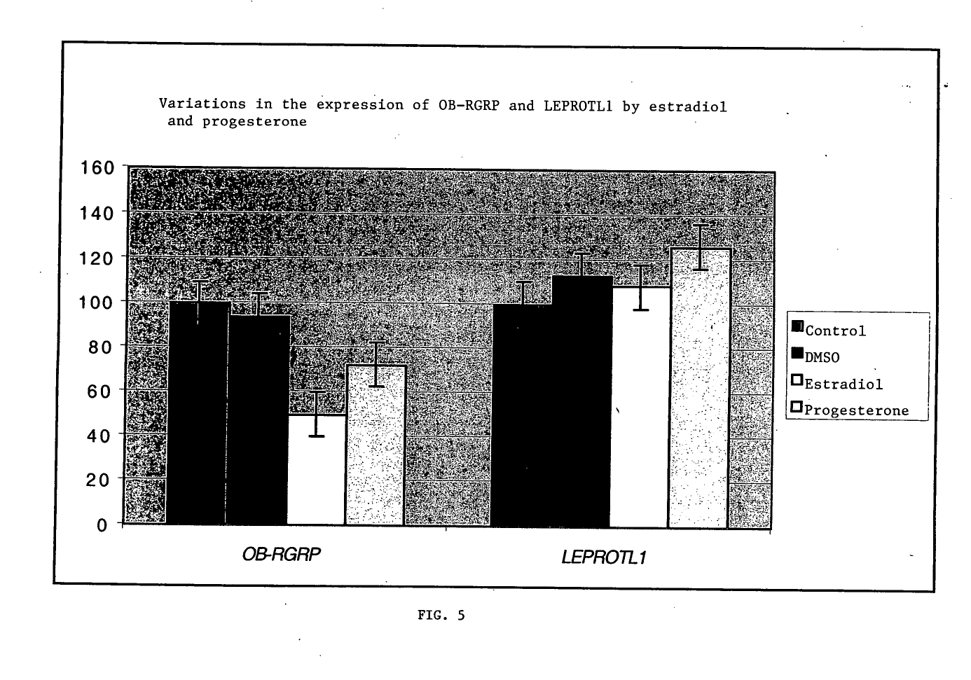

Expression values of OB-RGRP (RNAm) and of LEPROTL1 (RNAm) (normalized by the RNAm's of b-actin and GAPDH) for the solvent treatment alone and the two hormonal treatments including the confidence interval at 95% are represented in FIG. 5.

Reductions of the expression of OB-RGRP (RNAm) by the two hormones and the augmentation of the expression of LEPROTL1 (RNAm) by progesterone are significant (p<0.05 for N=46 for OB-RGRP/b-actin or OB-RGRP/GAPDH, n=24 FOR LEPROTL1/b-actin or LEPROTL1/GAPDH). A reduction of the expression of OB-R(RNAm) by these same hormones has been described (P. S. Duggal et al., 2002, Reproduction 123 (6) 899-905; Kitawaki et al., 2001, Mol. Hum. Reprod. 7 (6): 567-72. The latter suggests that it is probably the promoter common to OB-RGRP and OB-R that is regulated by these hormones.

Regulation of these genes by the hormones and their role in the intracellular traffic of leptin might account for the human sexual dimorphism in the rate of adipose mass and the rate of circulating leptin (M. S. Hickey, et al., 1996, Biochem. Mol. Med. 59 (1): 1-6).

### EXAMPLE 4

### Expression of LEPROTL1 and OB-RGRP in the Brain and Fetus of Mice

Expression of LEPROTL1 (RNAm) by Northern blots (Y. Huang et al. 2001, Biochim. Biophys. Acta, 1517, 327-331) observed in numerous tissues is analogous to the expression of OB-RGRP (RNAm) (B. Bailleul et al., 1997, Nucleic Acids Res., 25, 2752-2758). We analyzed the expression of the RNAm of LEPROTL1 and OB-RGRP by hybridization in situ in the brain and fetus of the mouse.

The distribution of LEPROTL1 (RNAm) and OB-RGRP (RNAm) on cuts of brain and of fetus was studied by hybridization in situ by a technique previously described in detail using probes marked on the ³⁵S of antisense RNA recognizing the exons 3 and 4 of OB-RGRP (J. Mercer et al., 2000, J. Neuroendocrinol., 12, 649-655) or the 415 base pairs of LEPROTL1 (DNAc) of mice, generated by PCR using the following primers:

| | |
|---|---|
| 16 | |
| ACGAATTCACCGCCATGGCAGGCATC, | (SEQ ID NO. 20) |
| and | |
| ACTCTAGACCACTGCTGCCAGCTGAAG,. | (SEQ ID NO. 21) |

The two probes were hybridized on 20 μm adjacent coronal section of the mouse brain at the level of the hypothalamus (A; bregma 0.82 mm B; bregma 1.46 mm) or of the fetus of 13.5 days p.c. with the placenta (C).

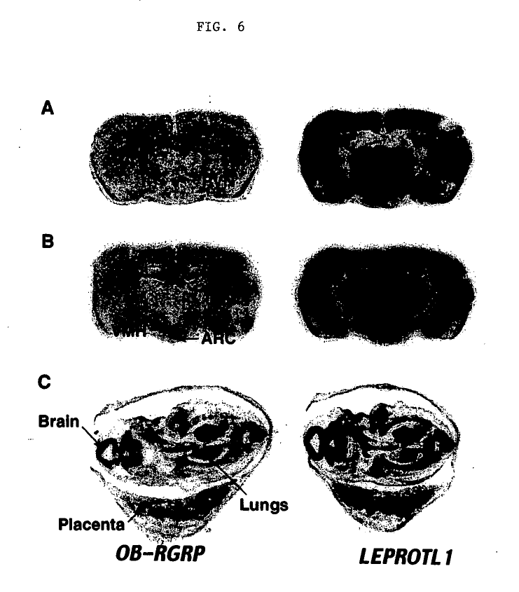

The audioradiographic signals generated by hybridization in situ with the LEPROTL1 probe are more intense than those observed with the OB-RGRP probe at the same rate of radioactivity and an identical specific radioactivity. Expression of LEPROTL1 is similar in distribution and abundance relative to the expression of OB-RGRP (FIG. 6). In the coronal sections across the hypothalamus, the two OB-RGRP and LEPROTL1 anti- sense probes hybridized for the number of defined structures with a weaker, broadly distributed intensity signal. The probe direction shows a weak and uniform signal (data not shown). The two RNAm's are broadly expressed in the hippocampus including the dentate gyrus (DG). Other structures and/tissues with distinct RNAm signals including the choroide plexus (C), the piriform cortex, the paraventricular nuclei (PVN), arc (ARC), ventromedian (VMH) (ventromedial nucleus of the hypothalamus), and the lateral hypothalamus (LH) (FIG. 6). In the areas of the central nervous system described above, localization on a microscope of the grains of silver of the test is identical to the neurons colored with toluidine blue.

The probes of LEPROTL1 and of OB-RGRP show a similar hybridization profile in the cuts of the fetus and the placenta. In the embryonic tissues, LEPROTL1 is more strongly expressed in the brain in development and the lungs, where an enhancement of the signal is observed for the OB-RGRP probe.

This data suggests that, as described for OB-RGRP, the expression of LEPROTL1 distributed broadly in the brain and the fetus with regions of stronger expression in some regions including the hypothalamic nuclei and the regions of the brain that express RNAm and the protein OB-R.

### EXAMPLE 5

### Modulation of the Expression of the Leptin Receptor on the Cellular Surface by the Protein OB-RGRP

Recombinant adenoviruses expressing a short isoform (ob-Ra) of the murine leptin receptor and the human protein OB-RGRP were constructed by homologous recombination. The leptin receptor expressed by this viral vector comprises an HA epitope at its N-terminal end. HeLa cells were co-infected with the virus expressing the receptor and increasing doses of virus expressing the protein OB-RGRP. The surface expression of the leptin receptor was quantified by biotinylation of the surface proteins, lysis of the cells, precipitation of the biotinylated material by spheres of streptavidin agarose and disclosure by immunoblot after separation of the proteins by electrophoresis in polyacrylamide gel. The leptin receptor was revealed with an anti-HA antibody. A portion of the cellular lysate was also subjected to the same analysis to determine the total quantity of receptors (surface+internal). The quantities of surface receptors were expressed by the ratio of the quantity of protein in the biotinylated fraction to the quantity of protein present in the total cellular lysates.

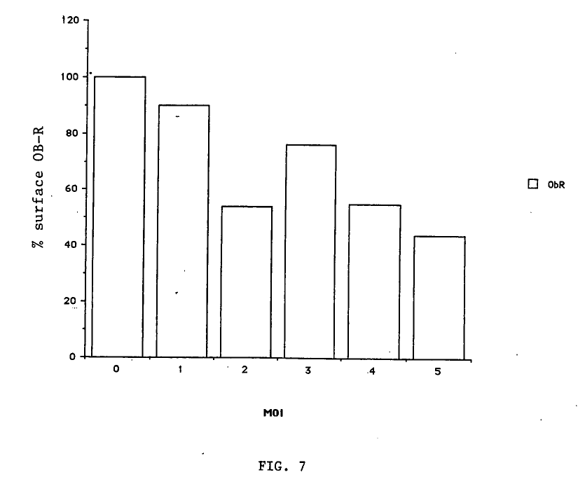

Surface expression of the leptin receptor is affected by overexpression of the protein LEPROTL1. Overexpression of the protein OB-RGRP induces a dose-dependent diminution of the surface expression of the leptin receptor (FIG. 7). The total quantity of leptin receptor (surface+internal) is not affected by overexpression of the protein OB-RGRP. These results demonstrate the specificity of the effects observed for surface expression of the leptin receptor and suggest that the protein OB-RGRP participates in regulation of surface expression of the leptin receptor.

1. A method for identifying compounds acting on weight gain or weight loss or on diabetes in a human or animal subject comprising:
measuring effect of the compounds on (a) expression of at least one of genes LEPROTL1 and OB-RGRP or a part of them and/or (b) intracellular transport up to a cellular membrane and/or (c) presence at the cellular membrane level and/or (d) internalization from the membrane of proteins coded by at least one of these genes or part of them.

2. The method according to claim 1, further comprising (i) contacting a compound to be tested with cells expressing at least one of the LEPROTL1 and OB-RGRP genes or a part of them, and (ii) measuring the effect of the compound on the quantity of proteins coded by at least one of the genes or fragments of them present at the level of the membrane of these cells and/or their internalization.

3. The method according to claim 1, further comprising marking endospanin and/or OB-RGRP or part of them coded by at least one of these genes or fragments of them.

4. The method according to claim 1, implemented in vitro on a model of cells in culture or in vivo in an animal.

5. The method according to claim 1, further comprising measuring expression and/or transport up to a surface of the membrane of the cells of OB-R or, in the case of an animal, measuring circulating leptin.

6. The method according to claim 4, wherein the cells or animal express(es) the genes LEPROTL1 and/or OB-RGRP or fragments of them in an endogenous manner which comprises genetically modification to express LEPROTL1 and/or OB-RGRP genes or fragments of them in the form of recombinant proteins.

7. The method according to claim 1, wherein the genes LEPROTL1 and/or OB-RGRP or fragments of them are human genes or genes of any other species having at least 80% homology with the human genes.

8. The method according to claim 1, wherein the genes LEPROTL1 and/or OB-RGRP or fragments of them are modified if they code for proteins that can be transported to the cellular membrane and then internalized.

9. The method according to claim 1, wherein the genes LEPROTL1 and/or OB-RGRP or fragments of them are in a form modified in such a manner as to produce marked proteins.

10. The method according to claim 9, wherein the marking consists of a peptidic marker.

11. The method according to claim 1, wherein the genes LEPROTL1 and/or OB-RGRP or fragments of them are modified and that they code for at least two of four transmembrane areas of these proteins and a peptidic marker in the form of fusion proteins.

12. The method according to claim 10, wherein the peptidic marker is inserted in the endospanin at the level of one of the two extracellular loops.

13. The method according to claim 10, wherein the peptidic marker is inserted at the level of its N or C terminal end of a truncated endospanin by a deletion and the deletion positions the terminal end and the marker in a luminal/extracellular position.

14. The method according to claim 1, wherein the effect of the compound to be tested is measured with the aid of an antibody directed against endospanin and OB-RGRP, a part or a marker thereof.

15. The method according to claim 1, wherein the effect of the compound to be tested is realized by a comparison of measurements on cells with and without their being placed in contact with the compound.

16. The method according to claim 1, wherein the effect of the compound to be measured is measured after or during a determined time of placing the cells in contact with the compound.

17. The method according to claim 1, wherein the effect of the compound to be tested on intracellular traffic of endospanin and/or OB-RGRP is also measured with an antibody directed against any surface membrane protein, a part or a marker thereof.

18. The method according to claim 11, wherein the peptidic marker is inserted in the endospanin at the level of one of the two extracellular loops.

19. The method according to claim 11, wherein the peptidic marker is inserted at the level of its N or C terminal end of a truncated endospanin by a deletion and that the deletion positions the terminal end and the marker in luminal/extracellular position.
